(19) United States
(12) Patent Application Publication
(10) Pub. No.: US 2009/0082828 A1
(43) Pub. Date: Mar. 26, 2009

(54) Leadless Cardiac Pacemaker with Secondary Fixation Capability

(76) Inventor: Alan Ostroff, San Clemente, CA (US)

Correspondence Address: SHAY GLENN LLP, 2755 CAMPUS DRIVE, SUITE 210, SAN MATEO, CA, 94403, US

(21) Appl. No.: 12/234,226

(22) Filed: Sep. 19, 2008

Related U.S. Application Data
(60) Provisional application No. 60/974,057, filed on Sep. 20, 2007.

(51) Int. Cl. A61N 1/375; A61N 1/362

(52) U.S. Cl. 607/36; 607/9

(57) ABSTRACT

The invention relates to leadless cardiac pacemakers (LBS), and elements and methods by which they affix to the heart. The invention relates particularly to a secondary fixation of leadless pacemakers which also include a primary fixation. Secondary fixation elements for LBS's may either actively engage an attachment site, or more passively engage structures within a heart chamber. Active secondary fixation elements include a tether extending from the LBS to an anchor at another site. Such sites may be either intracardial or extracardial, as on a vein through which the LBS was conveyed to the heart, the internal or external surface thereof. Passive secondary fixation elements entangle within intraventricular structure such as trabeculae carneae, thereby contributing to fixation of the LBS at the implant site.

### CROSS REFERENCE TO RELATED APPLICATIONS

This application claims priority to U.S. Provisional Application No. 60/974,057 filed Sep. 20, 2007, entitled “Leadless Cardiac Pacemaker with Secondary Fixation Capability”, which application is incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD OF THE INVENTION

The present invention relates to leadless cardiac pacemakers, and more particularly, to features and methods by which they are affixed within the heart.

### BACKGROUND

Cardiac pacing by an artificial pacemaker provides an electrical stimulation of the heart when its own natural pacemaker and/or conduction system fails to provide synchronized atrial and ventricular contractions at rates and intervals sufficient for a patient's health. Such antibradycardial pacing provides relief from symptoms and even life support for hundreds of thousands of patients. Cardiac pacing may also provide electrical overdrive stimulation to suppress or convert tachyarrhythmias, again supplying relief from symptoms and preventing or terminating arrhythmias that could lead to sudden cardiac death.

Cardiac pacing by currently available or conventional pacemakers is usually performed by a pulse generator implanted subcutaneously or sub-muscularly in or near a patient's pectoral region. Pulse generator parameters are usually interrogated and modified by a programming device outside the body, via a loosely-coupled transformer with one inductance within the body and another outside, or via electromagnetic radiation with one antenna within the body and another outside. The generator usually connects to the proximal end of one or more implanted leads, the distal end of which contains one or more electrodes for positioning adjacent to the inside or outside wall of a cardiac chamber. The leads have an insulated electrical conductor or conductors for connecting the pulse generator to electrodes in the heart. Such electrode leads typically have lengths of 50 to 70 centimeters.

Although more than one hundred thousand conventional cardiac pacing systems are implanted annually, various well-known difficulties exist, of which a few will be cited. For example, a pulse generator, when located subcutaneously, presents a bulge in the skin that patients can find unsightly, unpleasant, or irritating, and which patients can subconsciously or obsessively manipulate or “twiddle”. Even without persistent manipulation, subcutaneous pulse generators can exhibit erosion, extrusion, infection, and disconnection, insulation damage, or conductor breakage at the wire leads. Although sub-muscular or abdominal placement can address some concerns, such placement involves a more difficult surgical procedure for implantation and adjustment, which can prolong patient recovery.

A conventional pulse generator, whether pectoral or abdominal, has an interface for connection to and disconnection from the electrode leads that carry signals to and from the heart. Usually at least one male connector molding has at least one terminal pin at the proximal end of the electrode lead. The male connector mates with a corresponding female connector molding and terminal block within the connector molding at the pulse generator. Usually a setscrew is threaded in at least one terminal block per electrode lead to secure the connection electrically and mechanically. One or more O-rings usually are also supplied to help maintain electrical isolation between the connector moldings. A setscrew cap or slotted cover is typically included to provide electrical insulation of the setscrew. This briefly described complex connection between connectors and leads provides multiple opportunities for malfunction.

Other problematic aspects of conventional pacemakers are enumerated in the related applications, many of which relate to the separately implanted pulse generator and the pacing leads. By way of another example, the pacing leads, in particular, can become a site of infection and morbidity. Many of the issues associated with conventional pacemakers are resolved by the development of a self-contained and self-sustainable pacemaker, or so-called leadless pacemaker, as described in the related applications cited above.

Self-contained or leadless pacemakers or other biostimulators are typically fixed to an intracardial implant site by an actively engaging mechanism such as a screw or helical member that screws into the myocardium. Examples of such leadless biostimulators are described in the following publications, the disclosures of which are incorporated by reference: (1) U.S. application Ser. No. 11/549,599, filed on Oct. 13, 2006, entitled “Leadless Cardiac Pacemaker System for Usage in Combination with an Implantable Cardioverter-Defibrillator”, and published as US2007/0088394A1 on Apr. 19, 2007; (2) U.S. application Ser. No. 11/549,581 filed on Oct. 13, 2006, entitled “Leadless Cardiac Pacemaker”, and published as US2007/0088396A1 on Apr. 19, 2007; (3) U.S. application Ser. No. 11/549,591, filed on Oct. 13, 2006, entitled “Leadless Cardiac Pacemaker System with Conductive Communication” and published as US2007/0088397A1 on Apr. 19, 2007; (4) U.S. application Ser. No. 11/549,596 filed on Oct. 13, 2006, entitled “Leadless Cardiac Pacemaker Triggered by Conductive Communication” and published as US2007/0088398A1 on Apr. 19, 2007; (5) U.S. application Ser. No. 11/549,603 filed on Oct. 13, 2006, entitled “Rate Responsive Leadless Cardiac Pacemaker” and published as US2007/0088400A1 on Apr. 19, 2007; (6) U.S. application Ser. No. 11/549,605 filed on Oct. 13, 2006, entitled “Programmer for Biostimulator System” and published as US2007/0088405A1 on Apr. 19, 2007; (7) U.S. application Ser. No. 11/549,574, filed on Oct. 13, 2006, entitled “Delivery System for Implantable Biostimulator” and published as US2007/0088418A1 on Apr. 19, 2007; and (8) International Application No. PCT/US2006/040564, filed on Oct. 13, 2006, entitled “Leadless Cardiac Pacemaker and System” and published as WO07047681A2 on Apr. 26, 2007.

The site of attachment of leadless biostimulators is physically reinforced by a foreign body response that results in the growth of fibrotic tissue that further secures the leadless biostimulator at the attachment site. A high degree of success of attachment by such an approach notwithstanding, the potential of detachment of the leadless biostimulator from the implant site would represent an immediately serious event, as for example, a pacemaker lost from the right ventricle can exit the heart via the pulmonic valve and lodge in the lung. Leadless or self-contained biostimulators would benefit from mechanisms and methods for “secondary fixation” of the device within the heart, or more generally, features that in the event of failure of the primary fixation to the implant site would prevent escape of the pacemaker into the circulation downstream from the heart.

### SUMMARY OF THE INVENTION

The invention relates to a leadless cardiac pacemaker, a device more generally referred to as a leadless biostimulator (LBS), which includes a primary fixation element and a secondary fixation element. The invention also relates to methods of implanting a biostimulator with such a secondary fixation feature, and more generally to methods for retaining a leadless biostimulator in the heart in the event that the biostimulator is dislodged from its site of primary fixation.

With regard to embodiments of a leadless biostimulator with both primary and secondary fixation features, embodiments of the primary fixation element may be either active or passive; active elements typically requiring an active engagement of the element to a portion of the heart on the part of the user implanting the LBS and/or an active or at least minimally invasive engagement of heart structure, and the passive embodiments not so-requiring. Embodiments of the secondary fixation element or assembly may also be characterized as active or passive. Exemplary embodiments of active forms of a secondary fixation assembly include an anchor and a tether, the tether connecting the LBS to the anchoring site, and the anchoring site actively engaging heart or vascular structure. Embodiments of passive types of fixation include entangling elements connected to the LBS which become entangled in structural features within the heart chamber where the LBS is implanted.

Embodiments of a leadless biostimulator typically include a primary fixation element adapted to affix the biostimulator to a primary fixation site on a heart wall within a heart chamber; and a downstream vascular escape prevention assembly adapted to prevent an escape of the biostimulator in the event of it being dislodged from the implant site in a chamber of the heart. Other components of the leadless biostimulator include a power source adapted to be disposed within a human heart chamber, an electrode in electrical communication with the power source and adapted to be placed in contact with tissue within the heart chamber, a controller adapted to be disposed within the heart chamber and to control delivery of electrical energy from the power source to the electrode. Some embodiments of the leadless biostimulator include a housing within which the power source, the electrode, and the controller are disposed. Some embodiments of the biostimulator may be adapted for implantation in the right ventricle or the left ventricle of the heart; in other embodiments, the biostimulator may be implanted in the left or right atrium of the heart.

Some embodiments of a leadless biostimulator have a downstream vascular escape prevention assembly that includes one or more entangling elements adapted to entangle within heart structure at one or more secondary fixation sites within the chamber of the heart. In some of these embodiments, the one or more entangling elements may include any of tines, hooks, or chains. Typical embodiments of entangling elements are adapted to extend radially outward beyond the diameter of the biostimulator, particularly after the biostimulator is implanted. Some of the entangling element embodiments are at least 5 mm in length. Some of the entangling element embodiments extend outward from the biostimulator at a proximal-facing angle that ranges from about 10 degrees to about 90 degrees from the axis of the biostimulator. Some of the entangling element embodiments such as tines are configured as any of straight tines, curvilinear tines, or convoluted tines.

Some of the entangling element embodiments are adapted to be rotatable with respect to the biostimulator, as for example, they may be mounted on a rotatable collar encircling the main axis of the biostimulator. Some of the entangling element embodiments are configured such that they are distally-collapsible around the periphery of the biostimulator. When collapsed, typical embodiments of collapsible entangling elements are configured to be substantially contained within a maximal diameter of the biostimulator, or add a minimal increment to such maximal diameter.

Some embodiments of a the leadless biostimulator have a downstream vascular escape prevention assembly that includes a tether and an anchor, the tether connecting the assembly and the anchor to each other, and the anchor adapted to anchor at a secondary attachment site. In these embodiments, the anchor may include any of a screw, a hook, a clip, a stent, a cage, or a barb to attach the biostimulator to the secondary attachment site. The attachment site to which the anchor plus tether embodiments of secondary fixation to which the anchor is adapted to affix may be any of an intracardiac site, an intravascular site, or an extravascular site. In some embodiments, the intracardiac site is a septal wall of the heart. In other embodiments, the intravascular site is located within a vessel through which the biostimulator was delivered to the heart. Such vessels may include, for example, any of the femoral vein or the inferior vena cava. In some of these embodiments, the tether of the biostimulator is formed from two segments secured together with a clip. In other embodiments, an extravascular site may include the external periphery of a vessel through which the biostimulator was delivered to the heart. In these embodiments, the tether is typically adapted to be threaded through the vessel wall and to be attached to an anchor, the anchor including, by way of example, any of a partial cylinder, a plate, or a ball. In some anchor-plus-tether embodiments, the connection between the anchor and the tether, or between the tether and the biostimulator may include intervening or connective elements.

In some embodiments of a leadless biostimulator, the anchor may include one or more electrodes for biostimulation, wherein the tether itself is electrically conductive. In some embodiments, the tether may include any of single strand wire, multistranded wire, monofilament suture thread, or multistrand suture thread. In some embodiments, a tether or any of the anchor itself, or entangling elements may include any of a biodegradable material or an antithrombogenic agent.

Some embodiments of a leadless biostimulator may include one or more soluble coverings configured to encapsulate any of the primary fixation element or the secondary fixation element. Some embodiments of the soluble covering may include biocompatible materials, such as, merely by way of example, a polymer (such as polyvinylpyrrolidone), a protective sugar (such as mannitol), or a protective salt. In typical embodiments that make use of a soluble covering that is useful in deployment of the device, the soluble covering secures the secondary element in a collapsed configuration.

As mentioned above, embodiments of the invention also include a method for retaining a leadless intracardiac biostimulator in the heart in the event of dislodgement from a primary fixation site. In some embodiments, the method including the step of entangling an element of the biostimulator within the heart structure at a site within a heart chamber, such entanglement being sufficient to retain the biostimulator within the cardiac chamber. Embodiments of this method may include entangling the biostimulator or an element of the biostimulator within heart structures such as trabeculae in either the left or right ventricle. In another aspect, some embodiments of the invention include preventing escape of the biostimulator into a downstream vascular site, such as the aorta, if preventing escape from the left ventricle, or the pulmonary artery, if preventing escape from the right ventricle.

Some embodiments of a method for retaining a leadless intracardiac biostimulator in a heart in the event of dislodgement from a primary fixation site include anchoring the biostimulator with a tether to a secondary anchoring site, the tether being of appropriate length (e.g., sufficiently short) to prevent substantial movement into a downstream vascular from a biostimulator implant site in a heart chamber. In some aspects, anchoring the biostimulator with a tether includes anchoring with a tether of appropriate length to retain the biostimulator within the heart chamber.

In some embodiments, anchoring the biostimulator with a tether includes attaching the tether to an anchor at the secondary fixation site. Such attaching may include attaching the tether to the secondary fixation site with any of a screw, a hook, a clip, a stent, a cage, or a barb.

In various aspects, anchoring the biostimulator to a secondary anchoring site can include anchoring to either an intracardiac site or an extracardial site. In some embodiments, anchoring to an extracardial site includes anchoring to a site on a vessel through which the biostimulator was delivered to the heart. Also, in these embodiments, the anchoring site may be on either an internal or an exterior surface of the vessel.

Some embodiments of a method for retaining a leadless intracardiac biostimulator in a heart in the event of dislodgement from a primary fixation site that include anchoring the biostimulator with a tether to a secondary anchoring site include combining two tethers to form a single tether. Such a method of forming a single combined tether from two original tethers can include inserting a biostimulator attached to a first tether into an entry site in the vasculature, advancing the biostimulator to an intracardial implant site, and implanting the biostimulator at that site, inserting an anchor attached to a second tether into the entry site in the vasculature, advancing the anchor to a secondary anchoring site, and implanting the anchor at that site, and engaging the tether of the biostimulator and the tether of the anchor within a slidable clip at the vascular entry site to form a combined tether. Embodiments of this method may further include adjusting the length of the combined tether by slidably advancing the clip within the vasculature toward secondary anchoring site, and securing the first tether and the second tether at the clip so that no further sliding can occur. More specifically, adjusting the length of the combined tether may include adjusting the length such that there is an appropriate level of slack between the anchoring site and the biostimulator.

In another aspect, rescuing a leadless biostimulator dislodged from its primary fixation site may include a user grasping any portion of a secondary fixation element with a tool, and withdrawing the dislodged biostimulator from the heart chamber in which it was implanted.

Embodiments of the invention may further include fixation elements that are redundant, ancillary, or supportive of primary fixation, by, for example, minimizing movement of the biostimulator at the implant site. Such movement may include, for example, undesirable pitch, or yaw, or roll. Some of the embodiments may include rigid elements that are attached or connected to a primary fixation element on one end, and seated into or against heart structure on the other end. Some of these embodiments, which mainly serve in a primary fixation capacity, may further provide a secondary fixation.

### BRIEF DESCRIPTION OF THE FIGURES

FIG. 1A shows a leadless biostimulator at an implant site at the apex of the right ventricle. FIG. 1B is an expanded view of encircled portion of FIG. 1A, showing the biostimulator in the midst of trabeculae, and fixed at the implant site by a primary fixation helix that embeds in the myocardium, and secondarily fixed by a distally-situated set of entangling elements on a rotatable collar.

FIG. 2 shows a leadless biostimulator, with multiple depictions thereof for purposes of illustrating various implantation sites, as implanted at the apex of the right ventricle and at other sites on the ventricle wall.

FIG. 3A shows an embodiment of a leadless biostimulator with passive, trabeculae-engaging primary fixation elements on the distal end, facing distally, and also having secondary fixation entangling elements at the proximal end of the biostimulator, facing proximally. FIG. 3B shows the biostimulator of FIG. 3A in situ, at an implant site at the apex of the right ventricle.

FIGS. 4A-4D show an embodiment of a leadless biostimulator with an active primary fixation element at its distal end, as do FIGS. 5 and 7. FIG. 4A shows the leadless biostimulator in a deployment tube for insertion, with secondary fixating tines distally collapsed within the deployment tube. FIG. 4B shows an embodiment similar to that of 4A, but with the tines collapsed proximally within the deployment tube. FIG. 4C shows the biostimulator after deployment, with the tines released and projecting outward. FIG. 4D shows an end view of the biostimulator with the tines projecting outward.

FIG. 5 shows a leadless biostimulator with another embodiment of an active primary fixation element, in this case a distally mounted and distally-directed helical element that can rotatively engage the cardiac wall and affix to it.

FIG. 6A shows an embodiment of a leadless biostimulator with a passive primary fixation element having four tines. FIG. 6B shows an end view of the biostimulator.

FIGS. 7A-7C show an embodiment of a leadless biostimulator with an active primary fixation element at its distal end, in a series of views similar to that of FIG. 4. The embodiment depicted here differs from the embodiment depicted in FIG. 4 by having more tines, and by the tines having a knob at their distal end. FIG. 7A shows the leadless biostimulator in a deployment tube for insertion, with distally-directed primary anchoring tines collapsed within the deployment tube. FIG. 7B shows the biostimulator after deployment with the tines released and projecting outward. FIG. 7C shows an end view of the biostimulator with the tines projecting outward.

FIG. 8 shows an embodiment of a leadless biostimulator with a primary fixation system at the distal end and a pair of clip-like secondary fixation elements on a rotating collar mounted on the midsection of the biostimulator.

FIGS. 9A and 9B show an embodiment similar to that of FIG. 8, but with the fixation elements mounted on the proximal portion of a biostimulator. FIG. 11B depicts the biostimulator as it engages trabeculae in a heart chamber.

FIG. 10A-10E show an embodiment of a leadless biostimulator with both a primary fixation element and secondary fixation elements at the distal end of the stimulator, the secondary elements comprising proximally biased knobbed tines. FIG. 10A shows the biostimulator in a deployment tube, FIG. 10B shows the biostimulator being ejected from the deployment tube within a heart chamber; FIG. 10C shows the biostimulator affixed to an implant site; FIG. 10D shows the biostimulator being captured by a retraction tube; and FIG. 10E shows the biostimulator having been drawn up into the retraction tube.

FIGS. 11A-11C show an embodiment of a leadless biostimulator with secondary fixation elements in the form of nibs arranged in a helical pattern along the mid- and distal portions of the biostimulator, and secondary fixation elements in the form of outwardly projecting trabeculae entangling tines at the proximal portion of the biostimulator. FIG. 11A shows the biostimulator in isolation; FIG. 11B shows the biostimulator emerging from a deployment tube, the secondary fixation elements still within the tube; FIG. 11C shows the biostimulator as it has emerged from the deployment tube, the secondary fixation elements having engaged the trabeculae, and the proximally-located secondary fixation tines now unfolded.

FIGS. 12-16 show various embodiments of a leadless biostimulator, each having primary fixation system, either passive (as illustrated by FIGS. 12 and 14) or active (as illustrated by FIGS. 13, 15, and 16) at the distal end of the biostimulator, and each biostimulator also having at least one secondary fixation system comprising entangling elements on the proximal and/or distal portion(s) of the biostimulator.

FIGS. 17A-17C show a series of embodiments of a leadless biostimulator, each with an active primary fixation element at the distal end of the biostimulator, and each with a pair of passive secondary fixation elements in the form of an entangling set of tines at the proximal end and distal end of the biostimulator. The entangling elements are biased and collapsible proximally, and have varied proximal-facing angles when expanded as shown. The extremities of the tines of FIG. 17A form an angle of about 90 degrees from the main axis of the biostimulator; the extremities of the tines of FIG. 17B form an angle of about 45 degrees, and the extremities of the tines of FIG. 17C form an angle of about 10 degrees.

FIGS. 18A-18B show an embodiment of a leadless biostimulator with an entangling set of tines at the proximal portion of the biostimulator that are configured to serve as secondary fixation elements. FIG. 18A shows the tines collapsed distally against the periphery of the biostimulator and secured in the collapsed position by a soluble capsule. FIG. 18B shows the tines expanded into their deployed position, after the soluble capsule has dissolved.

FIGS. 19A and 19B show an embodiment of a leadless biostimulator with an entangling set of tines at the proximal portion of the biostimulator that serve as secondary fixation elements and a primary fixation element in the form of a set of distally-mounted proximally angled tines. FIG. 19A shows both sets of tines collapsed proximally against the periphery of the biostimulator and secured in the collapsed position by soluble capsules encasing both the proximal and distal ends of the biostimulator. FIG. 19B shows both sets of tines expanded into their deployed position, after the soluble capsule has dissolved.

FIG. 20 shows an embodiment of a leadless biostimulator with a primary fixation element on the distal end, and secondary fixation elements in the form of proximally-facing entangling tines mounted on a rotatable collar encircling the biostimulator. The rotatability of the collar allows the body of the leadless biostimulator to rotate while a primary fixation element (such as a helix) engages the heart wall without interference from the secondary fixation element as it becomes entangled and its rotational movement stopped.

FIG. 21A-21E shows several embodiments of entangling elements for secondary fixation of a leadless biostimulator, the entangling elements being generally knobbed, ringed, or beaded along a flexible spine, or linked together as in a chain.

FIGS. 22A-22D show various fishhook-modified examples of secondary fixation tines. FIG. 22A shows a leadless biostimulator with three fishhook-modified tines mounted on a rotatable collar at the proximal portion of the device. FIG. 22B shows a similar leadless biostimulator embodiment, but with double fishhooks on each tine. FIG. 22C shows a leadless biostimulator with a single modified tine mounted on a rotating cap at the proximal end of the device, the tine modified into a triple fishhook configuration. FIG. 22D shows a similar leadless biostimulator with multiple triple-hook modified tines.

FIGS. 23A and 23B show an example of a secondary fixation approach in the form of ring-shaped entangling elements at the ends of tines with a distal-facing angle. Some examples of embodiments of this general form, when deployed, may form a lateral dimension sufficiently wide that movement through the pulmonic valve is prevented in the event of detachment from the primary fixation site. FIG. 23A depicts this embodiment compressed within a deployment tube, and FIG. 23B depicts the embodiment in a deployed state, the entangling or through-passage blocking elements in their expanded configuration.

FIGS. 24A and 24B show an example of a secondary fixation approach which is similar to that represented by the embodiment shown in FIG. 23, in that entangling elements may occupy sufficient width that they preclude movement of a biostimulator loosed from its primary attachment site through the pulmonic valve. FIG. 24A shows the biostimulator in a deployment tube; FIG. 24B shows the biostimulator in its post-deployment expanded configuration.

FIG. 25 shows an embodiment of a leadless biostimulator in situ at the apex of the right ventricle, further showing non-cardiac vascular sites for anchoring a tether, the sites occurring along the length of the inferior vena cava and the femoral vein, an exemplary vascular path through which the biostimulator may be implanted.

FIG. 26 shows an embodiment of a leadless biostimulator in situ at the apex of the right ventricle, and a tether connecting the biostimulator to an anchor located at the left femoral vein.

FIG. 27 shows an embodiment of a leadless biostimulator in situ at the apex of the right ventricle, and a tether connecting the biostimulator to an intraluminal stent located within the inferior vena cava.

FIGS. 28A-28D show an embodiment of a leadless biostimulator in situ at the apex of the right ventricle with an alternatively-embodied tether connecting the biostimulator to an anchoring site located within the inferior vena cava. More particularly, 28A-28D depict a method by which such a tether may be formed. FIG. 28A shows an early stage in the method, wherein a tether proximally connected to the leadless biostimulator emerges through a site in the femoral vein, and a second tether proximally connected to an anchoring site along the length of the inferior vena cava also emerges from the same site. In FIG. 28B, both tethers have been enclosed within a slidable clip, the clip is shown within the femoral vein and is being advanced proximally toward the anchoring site. In FIG. 28C, the clip has been proximally advanced to the locale of the anchoring site, and the portions of each tether distal to the clip are about to be cut off and removed, to form an integrated single tether. In FIG. 28D, the tether formation is complete; it has become situated substantially proximal to the anchoring site and extends proximally to the biostimulator residing in the heart, the clip remaining at the junction of the formerly separate tethers.

FIG. 29 shows an illustrative embodiment of a leadless biostimulator with multiple secondary fixation assemblies, each including an anchor tethered to the biostimulator, the anchors located at various wall sites within the right ventricle, the multiple sites shown for purposes of illustration, any single embodiment not necessarily having more than one tethered anchor for secondary fixation.

FIGS. 30A-30D show an embodiment of a leadless biostimulator in situ at the apex of the right ventricle with an alternatively-embodied tether connecting the biostimulator to an anchoring site located within the right ventricle. FIG. 30A shows an early stage in the method, wherein a tethered biostimulator with an attached tether has been implanted in a ventricle, and a secondary anchor with a secondary tether has been implanted in the same ventricle. Both tethers exit the heart emerge from an entry/exit site in the femoral vein (not shown). In FIG. 30B, both tethers have been enclosed within a slidable clip, the clip is shown at a stage where it has been proximally advanced from the entry site to a location in the inferior vena cava and is about to enter the heart, more specifically the right ventricle. In FIG. 30C, the clip has been proximally advanced to the locale of the secondary fixation anchoring site, and the portions of each tether distal to the clip are about to be cut off and removed, in order to form an integrated single tether. In FIG. 30D, the formation of the integrated tether is complete; and it connects the biostimulator directly to the anchoring site on the ventricular wall.

FIG. 31 shows an embodiment a leadless biostimulator with a flex member that has expanded into a substantially rigid member that seats into the subannular shelf of the right ventricle.

FIGS. 32A-32C show the deployment of the embodiment depicted in FIG. 31. FIG. 32A shows the flex member folded within a deployment tube about to emerge. FIG. 32B shows the flex member nearly completely emerged from the deployment tube, one of the ends seated against the subannular shelf, and the other seated against the proximal end of a leadless biostimulator at an implant site. FIG. 32C shows the expanded flex member in place.

### DETAILED DESCRIPTION OF THE INVENTION

As introduced in the background, leadless biostimulators (LBS's), also known as leadless cardiac pacemakers, for all their advantageous features over conventional pacemakers, could include as part of their profile a risk of loss into the downstream vasculature in the event of dislodgment from their site of primary fixation, were it not for the solution provided by embodiments of this invention. This invention provides various downstream vascular escape prevention methods and assemblies employing, e.g., “secondary fixation” in order to distinguish this form of attachment or fixation from “primary fixation”. In this context, primary fixation generally refers to an attachment or fixation of a cardiac pacemaker to an intracardial implant site (or primary fixation site) such that at least one of the electrodes of the biostimulator stably remains in intimate contact with that site on the myocardium. In contrast, secondary fixation generally refers to an element or assembly that retains within the heart chamber a biostimulator that has become loose from its implant site, or prevents the biostimulator from moving any substantial distance into the vasculature downstream from the chamber in which it was implanted, when it has become dislodged.

Retention within the heart chamber thus involves the engagement of one or more secondary fixation elements, at one or more secondary fixation sites. The nature and location of secondary fixation sites may vary in accordance with the nature of the secondary fixation element or the downstream vascular prevention assembly embodiments. Some secondary fixation embodiments include elements that entangle themselves passively within or amongst structural features within the heart chamber, and thus these secondary sites are located within the heart chamber where the device is implanted. These intracardial entangling fixations may be temporary or transient, as the engagement of an entangling element with structure may include sliding or twisting, as examples of transient engagement. In some embodiments or instances, the secondary fixation brought about by an entangling element may effectively become as secure as a typical primary fixation site, either by the effectiveness of entanglement, or by fibrotic process of heart tissue that engages the entangling element. Other embodiments of secondary fixation assemblies, as described herein, may include assemblies comprising an anchor and a tether, the tether connecting the leadless biostimulator to the anchoring site. The anchoring site for these embodiments may be considered the secondary fixation site, and such sites may be intracardial or extracardial. The tether of these embodiments may be composed of any suitable material or mixture of materials, such as, by way of example, single-stranded wire, multi-stranded wire, monofilament suture thread, or multi-stranded suture thread.

Some tether embodiments, as well as other components of secondary fixation elements, may also include an anti-thrombogenic agent to discourage them from becoming a clot-forming nucleus. In some embodiments of the LBS and associated methods of use, the acute phase following implantation is of particular significance in that during that time, the initial period of days or weeks following implantation, the primary fixation becomes more secure, as for example, as a result of the growth of fibrotic tissue envelopes the implant site. Accordingly during that time, the secondary fixation is of particular importance because of the relative vulnerability of the primary fixation. Further, accordingly, in some embodiments it may be appropriate for the tether to include biodegradable materials that degrade over time, after the acute and vulnerable phase has passed. By a similar rationale, it may be appropriate, in some embodiments, for entangling elements or secondary anchors include biodegradable materials.

Secondary fixation embodiments may vary with regard to the extent to which they re-enforce, assist, support, provide redundancy, or protect the primary fixation method or element. Some embodiments of secondary fixation may serve in one or more of these recited primary fixation-related capacities, either minimally or significantly. Other embodiments for secondary fixation elements or assemblies may provide no substantial contribution to the primary fixation function, and function entirely in their secondary fixation capacity when called upon in the event of failure of the primary fixation.

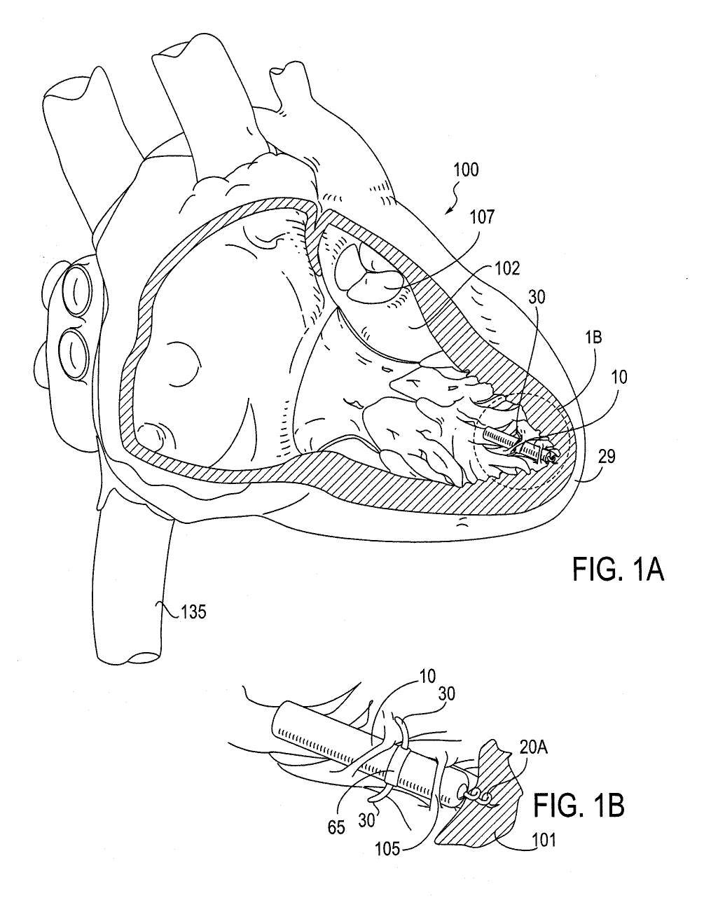

The U.S. patent publications listed in the background above describe and depict two basic types of primary fixation elements. One embodiment of a primary fixation element is a helix (e.g., FIG. 1A of US 2007/0088418) that may be screwed directly into the myocardium to form a very stable and secure fixation. The screwable helix approach to primary fixation may be considered “active” in that it entails a screwing action to seat it, and it is at least to some extent invasive of the myocardium. A second embodiment of a primary fixation element described therein includes a small set of tines (e.g., FIG. 1B of US 2007/0088418) that may be used alone or in combination with a screwable helix, and which are designed particularly to establish lateral stability on the myocardial surface. The primary fixating tines may be considered relatively “passive”, in comparison to the actively engaging screwable helix, as the engagement of the tines to the surface does not involve a screwing action, and the engagement is minimally invasive of the surface of the myocardium. Primary fixating tines typically do not extend or do not substantially extend beyond the diameter profile of the biostimulator, typically being less than 5 mm in length. Further, depending on the embodiment and the nature of the engagement of the primary fixating site, the times may be directed at an angle that varies between proximal and distal. The fixation provided by these tines may serve as a stand-alone fixation element, but may also be used in conjunction with a helix, in which case they may be understood to be a redundant, back-up, or supportive form of primary fixation. Both types of primary fixation elements are subject to fibrotic overgrowth, as mentioned in the background, which further supports the fixation of the LBS at the attachment site.

The secondary fixation elements described herein perform a fail-safe function by, after failure of primary fixation, preventing loss of a dislodged LBS from a ventricle in which it's implanted, and they may further, in some embodiments, support stability of the LBS at the implant site. For example, if an LBS implanted in the right ventricle were to dislodge and exit the ventricle, it would leave through the pulmonic valve and lodge in the lungs. If an LBS implanted in the left ventricle were to exit the ventricle, it would enter the aorta and move into the general circulation, or the brain. A function of secondary fixation is to prevent occurrence of these catastrophic events should primary fixation fail. Some embodiments of the secondary fixation elements effectively retain a dislodged LBS within the ventricle, and other embodiments may allow exit from the ventricle for a very short distance but stop any substantial downstream movement. Dislodgment or detachment of an LBS from its implant site, even with loss from the ventricle and adverse downstream consequences being prevented, is nevertheless a serious medical emergency, and the loosed LBS needs to be retrieved. Thus, another benefit and function of the secondary fixation element is that it may contribute to the feasibility of a retrieval procedure, by providing an element easily graspable by a retrieval tool.

As with primary fixation elements, secondary fixation elements may be active (or actively-applied) or passive (or passively-engaging). Active secondary fixation elements include a tether that connects the LBS to an anchor at a secondary site, the anchor being a secure attachment made by active engagement of a portion of the heart or engagement at an extracardial site. Passive secondary fixation embodiments include elements that hook, snag, or otherwise entangle within intrachamber structural features of the heart, but they are substantially non-invasive of heart structure, nor are they actively seated during implantation of the LBS. Anatomical heart structure in the chamber in which the elements entangle includes connective tissue structures generally referred to as trabeculae cameae that are prominent in ventricles, and may also include ridges in the myocardium, and may also include tissue with a mix of fibrous and muscular tissue. Trabeculae cameae may be referred to simply as trabeculae in the cardiac context; the structures are attached to the chamber wall and vary in form, appearing as ridges, flaps, and cords.

Embodiments of passive secondary fixation elements or entangling elements are typically closely associated with the body of the LBS, i.e., they are integral with the body of the LBS, directly attached to it, or mounted on a rotatable collar encircling the LBS. A typical embodiment of an entangling element is a set of one or more tines projecting outwardly from the body of the LBS, as described and depicted in detail below. In some embodiments, tines may include features that further provide engaging or particularly entangleable features, such as hooks, typically atraumatic hooks, or linked elements, such as for example, serial structures threaded together, or linked as in a chain. Tines may assume various forms; they may be straight or curved, they may project at various angles from the leadless biostimulator, and they may have a collapsible bias. Such collapsibility is advantageous for several reasons. In one aspect collapsibility reflects a flexible and compliant quality of the tines which is compatible with them being a structure that does not interfere with primary fixation. Further, the collapsibility has a bias that is typically proximally directed; this bias is consistent with the configuration of the landscape of the heart chamber that surrounds the primary attachment site. Collapsibility also provides for a structure that folds easily and closely around the body of the leadless biostimulator, which is a property advantageous for being accommodated by a delivery device, and further is compatible with being enclosed within a soluble capsule for deployment, and expanding outward to post-deployment configuration after dissolution of the soluble capsule. Typically, embodiments of tines project outwardly beyond the diameter of the leadless biostimulator to which they are attached, and typically, such tines are about 5 mm in length or longer.

Entangling elements may be attached to the LBS housing at any point along the body from proximal end to distal end, although they are generally not located at the distal-most point, because that locale is typically the location of a primary fixation element. The rotatable collar may be understood as a mount upon which tines may rotate around the main axis of the LBS body, or, from the complementary perspective, as a collar within which the LBS body may rotate. Rotation of the LBS body within the collar allows the body to turn as a screw, a movement that embeds a primary fixating helix into the myocardium while allowing the tines to come to rest as they encounter obstructing trabeculae in which they entangle.

The embodiments of leadless biostimulators 10 described herein and depicted variously in FIGS. 1-32 typically include at least two electrodes 68, a housing 60 that hermetically encloses the biostimulator's electrical components, a primary fixating element, either active 20A or passive 20B, and one or more secondary fixation elements. Embodiments of the secondary fixation elements may include forms such as entangling elements 30, or an assembly which includes a secondary fixation anchor 35 and tether 36 that tethers to the biostimulator to a secondary anchoring site 39. Secondary fixation entangling elements are typically mounted on a rotatable collar 65 that encircles the body or housing of the biostimulator, a feature that allows the entangling elements and the biostimulator to rotate with respect to each other. In order to focus illustrative attention on particular inventive features, such as secondary fixation elements, not every figure includes all features that may be present, or even must be present on a functional biostimulator. For example, all embodiments of biostimulator described herein should be understood to include at least two electrodes, even if not shown. Further, features depicted in the drawings of various embodiments of leadless biostimulators and fixation features may not be drawn to scale. Still further, a leadless biostimulator may be implanted in any heart chamber, atrium or ventricle, right or left side of the heart. A typical heart chamber into which a leadless biostimulator may be implanted is the right ventricle 102, and that is the exemplary and non-limiting implant site used herein for illustrative purpose.

In further regard to the at least two electrodes, one of the electrodes of the LBS must be in intimate contact with the myocardium. This electrode is typically located near the base of the helix or screw, and connects to the inside of the hermetic enclosure with a feed-through port. The other or second electrode may be the outer hermetic housing of the LBS body itself, a configuration that precludes the need for a second feed-through. There further may be a sensing advantage to masking the outer hermetic housing to only expose a ring around the can as the second electrode to simulate the electrode distances used in conventional bipolar pacing electrodes.

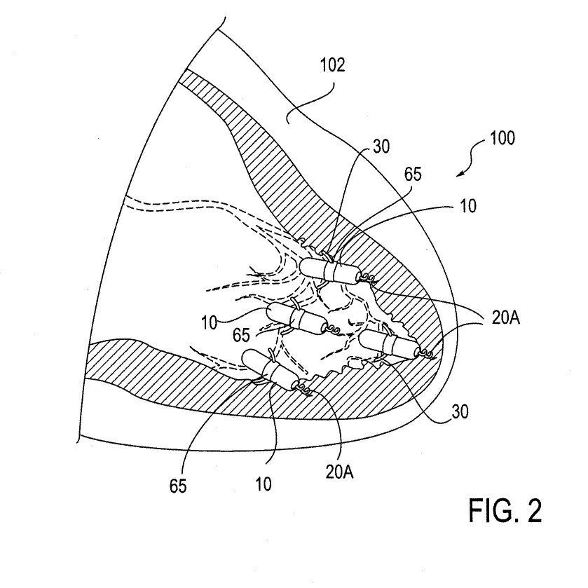

A leadless biostimulator 10 is shown in FIG. 1A at an implant site at the apex of the right ventricle 102 of a human heart 100. FIG. 1B provides an expanded view of encircled portion of FIG. 1A, showing the biostimulator in the midst of trabeculae 105, and fixed at the implant site 29 by a primary fixation helix 20A that embeds in the myocardium 101, and is secondarily fixed by a distally-situated set of entangling elements 30 on a rotatable collar 65. This embodiment can be understood to have been implanted through the use of delivery apparatus that screwed the primary fixation element 20A to engage the myocardium; as the LBS was being turned, the secondary fixation tines 30 were not forced to rotate because they are mounted on the aforementioned rotatable collar 65. The tines 30 can be seen to have a proximal bias, and to be proximally deflectable. By these properties, the tines have not interfered with the primary fixation, but have become entangled in the local trabeculae 105 such that if the primary fixation should fail, the secondary fixation represented by the passive engagement of the trabeculae by the tines would hold the biostimulator in the same general locale, and would prevent it from floating free and being swept into the downstream vasculature. FIG. 2 shows a leadless biostimulator 10, with multiple depictions thereof for purposes of illustrating various implantation sites, as implanted at the apex of the right ventricle 102 and at other sites on the ventricle wall. As depicted, a typical implant configuration is one where the distal portion of the LBS is nosed into the implant site 29, where the primary fixation element has engaged the myocardium.

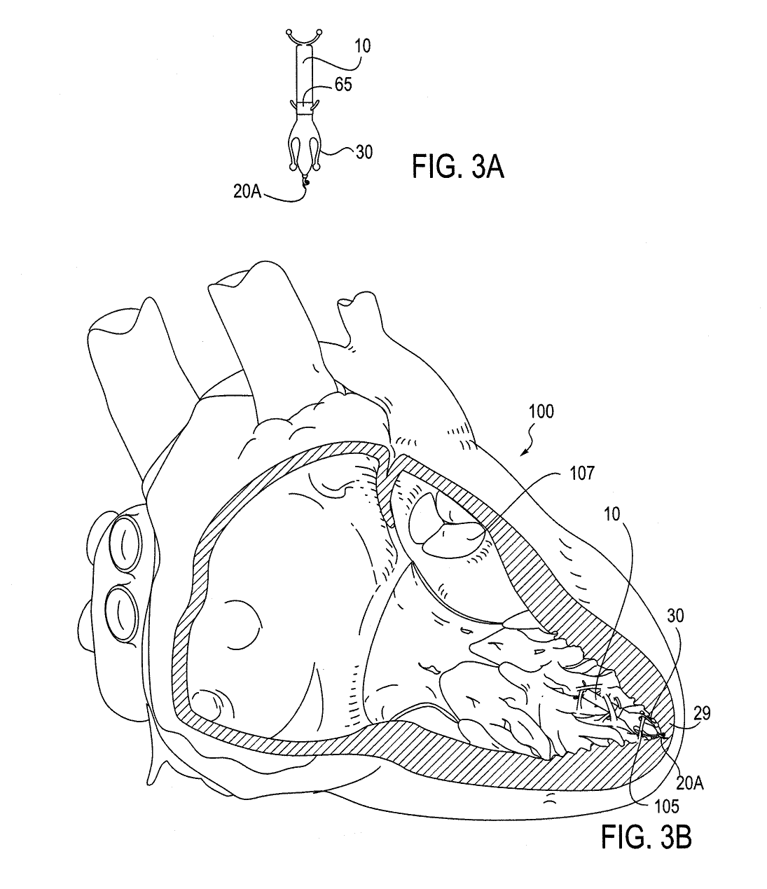

FIG. 3A shows another embodiment of a leadless biostimulator 10 with passive, trabeculae-engaging fixation entangling elements 30 on its distal end, facing distally but not projecting beyond the distal end of biostimulator, and also having secondary fixation entangling elements at the proximal end of the biostimulator, facing proximally. FIG. 3B shows the biostimulator of FIG. 3A in situ, at an implant site at the apex of the right ventricle. As depicted similarly in FIGS. 1A and 1B, the entangling secondary fixation elements have become entangled in local trabeculae 105. In this embodiment, with both tines situated at both the proximal and distal portions of the LBS, both sets of tines have become entangled in trabeculae. In another aspect of the method of secondary fixation, in some cases, entanglement of trabeculae by tine elements may be complete as the primary fixation is complete; in other embodiments, the entanglement may occur as a consequence of movement such as pitch or yaw that may occur during a prelude to dislodgment or after the unfortunate dislodgement of the LBS from its primary fixation site.

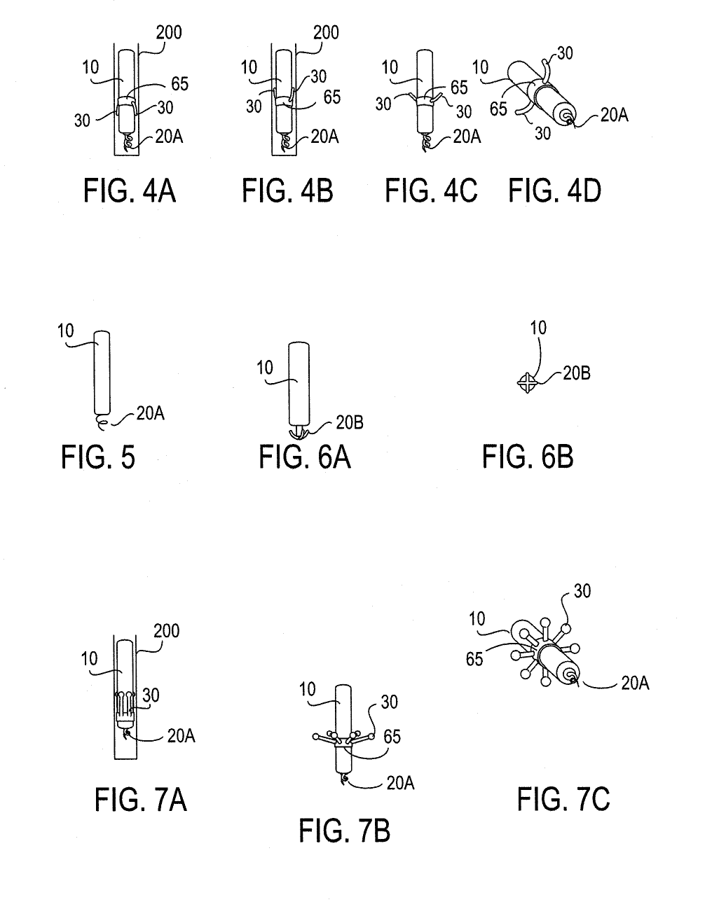

A series of embodiments of biostimulators with varied forms of primary fixation elements and passive secondary fixation elements are shown in FIGS. 4-24. Secondary fixation elements, typically entangling elements that engage trabeculae 105 are generally collapsible either distally or proximally so as to be conformable within the confines of a delivery apparatus 200. Once deployed, entangling elements may be generally swept back proximally, or swept forward distally, or project outward perpendicularly from the biostimulator body, depending on the location of the entangling elements on the body, and on the particular configuration of the element. FIGS. 4A-4D show an embodiment of a leadless biostimulator 10 with an active primary fixation element 20A, a helix, at its distal end. FIG. 4A shows the leadless biostimulator 10 in a deployment tube 200 for insertion, with secondary fixating tines distally collapsed within the deployment tube. FIG. 4B shows an embodiment similar to that of 4A, but with the tines collapsed proximally within the deployment tube. FIG. 4C shows the biostimulator 10 after deployment, with the tines released and projecting outward. FIG. 4D shows an end view of the biostimulator with the tines projecting outward.

FIG. 5 shows a leadless biostimulator 10 with another embodiment of an active primary fixating element 20A, in this case a distally mounted and distally-directed helical element that can rotatively engage the cardiac wall 101 and affix to it. This particular illustrated embodiment has no secondary fixation element or assembly, and is simply included to emphasize and isolate the location and nature of a typical primary fixation apparatus. Similarly, FIGS. 6A-6B shows an embodiment of a leadless biostimulator 10 with a passive primary fixating element 20B consisting of four tines. FIG. 6B shows an end view of the biostimulator 10. Primary fixating tines serve the function of primary fixation, and may be proximally- or distally-directed, typically at an angle of about 45 degrees with respect to the main axis of the biostimulator, and are typically smaller than secondary fixating tines, i. e., less than 5 mm in length, and not projecting substantially beyond the diameter of the body of the biostimulator. Other similar embodiments may include two or three tines, or more than four tines. The 45 degree angle exemplifies the angle of a typical embodiment, but other embodiments may be configured at angles that range between about 30 degree and about 60 degrees with respect to the main axis of the biostimulator.

FIGS. 7A-7C show an embodiment of a leadless biostimulator 10 with a passive secondary fixating element 30 at its distal end, in a series of views similar to that of FIG. 4. The entangling element embodiment 30 depicted here differs from the embodiment depicted in FIG. 4 by having more tines, and by the tines having a knob at their distal end, which may further enhance the ability of the tines to passively engage structure in the heart. The tines are mounted on a rotatable collar 65. FIG. 7A shows the leadless biostimulator 10 in a deployment tube 200 for insertion, with distally-directed secondary fixating tines 30 collapsed distally within the deployment tube. FIG. 7B shows the biostimulator after deployment with the tines 30 released and projecting outward. FIG. 7C shows an end view of the biostimulator with the tines 30 projecting outward.

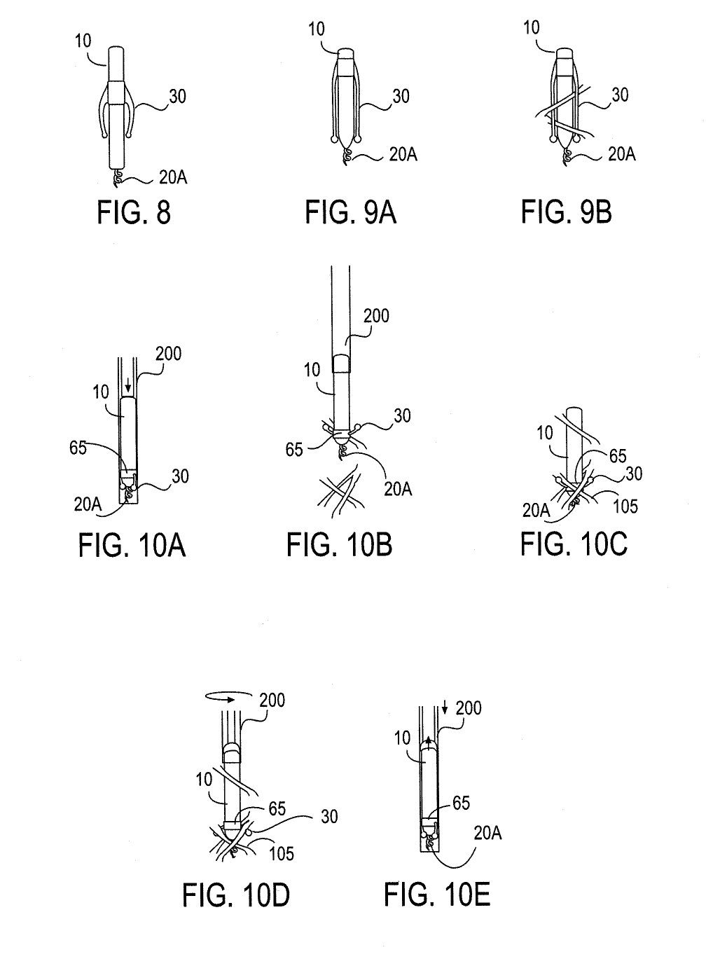

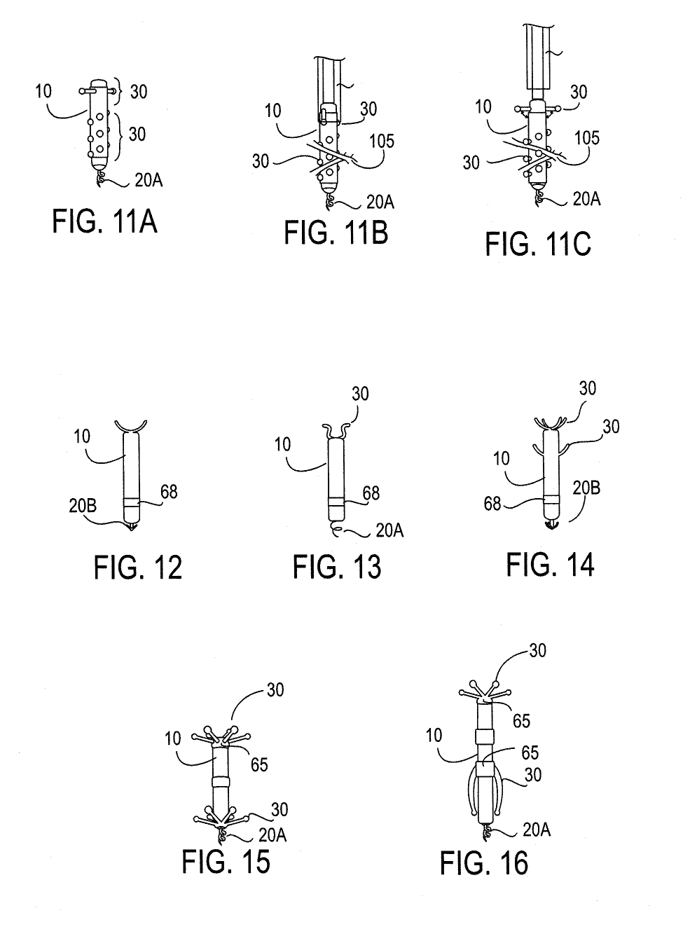

FIG. 8 shows an embodiment of a leadless biostimulator 10 with a primary fixation system 20A at the distal end and a pair of clip-like secondary fixation elements 30 with end-knobs on a rotating collar 65 mounted on the midsection of the biostimulator 10. FIGS. 9A and 9B show an embodiment of a leadless biostimulator 10 similar to that of FIG. 8, but with the secondary fixation elements 30 mounted on the proximal portion 12 of a biostimulator. FIG. 11B depicts the biostimulator 10 as it engages trabeculae 105 in a heart chamber.

FIGS. 10A-10E show an embodiment of a leadless biostimulator 10 with secondary fixation elements 30 at the distal end of the stimulator, the elements comprising proximally biased knobbed times, as well as an active primary fixating element 20A. FIG. 10A shows the biostimulator 10 in a deployment tube. FIG. 10B shows the biostimulator being ejected from the deployment tube 200 within a heart chamber. FIG. 10C shows the biostimulator affixed to an implant site 29 at its distal end, with the knobbed tines trapped within trabeculae 105. FIG. 10D shows the biostimulator being captured by a retraction tube 200, either by mechanical or vacuum means. In addition, FIG. 10E shows the biostimulator having been drawn up into the retraction tube, the secondary fixating tines having collapsed distally.

FIGS. 11A-11C show an embodiment of a leadless biostimulator 10 with secondary fixation elements 30 in the form of nibs arranged in a helical pattern along the mid- and distal portions of the biostimulator, and further secondary fixation elements 30 in the form of outwardly projecting trabeculae entangling tines at the proximal portion of the biostimulator. FIG. 11A shows the biostimulator 10 in isolation. FIG. 11B shows the biostimulator 10 emerging from a deployment tube 200, the secondary fixation elements still within the tube. FIG. 11C shows the biostimulator 10 as it has emerged from the deployment tube, the secondary fixation elements (helically arranged nibs) 30 having engaged the trabeculae, and the proximally-located secondary fixation tines 30 now unfolded.

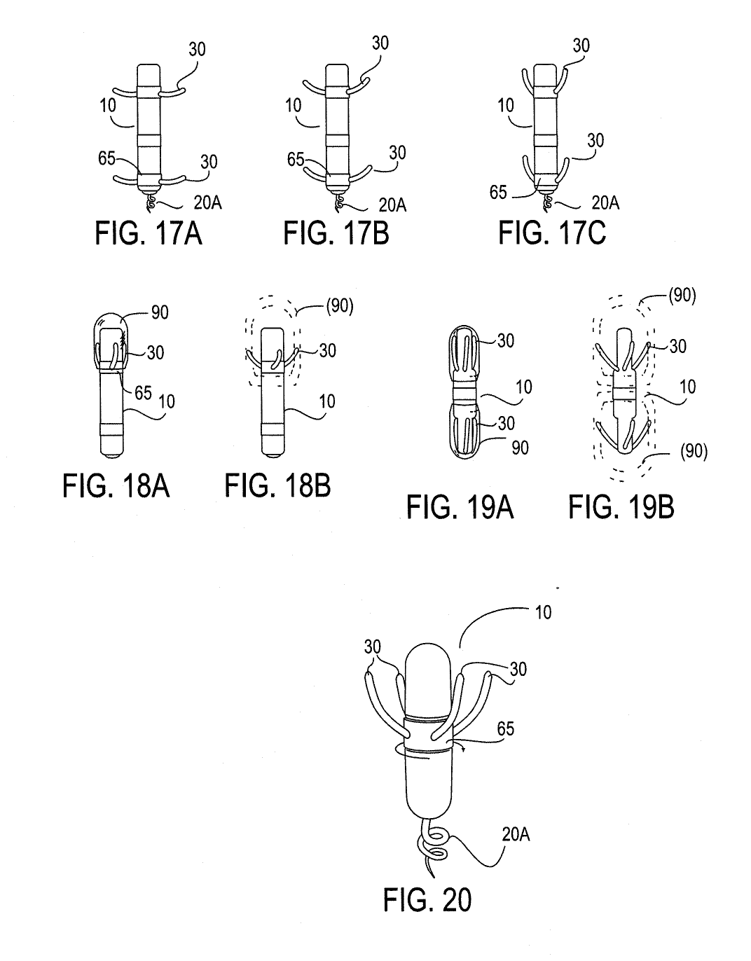

FIGS. 12-16 show various embodiments of a leadless biostimulator, each having primary fixation system, either passive (as illustrated by FIGS. 12 and 14) or active (as illustrated by FIGS. 13, 15, and 16) at the distal end of the biostimulator, and each biostimulator also having a secondary fixation system comprising entangling elements 30 on the proximal portion of the biostimulator. Thus, FIG. 12 shows a biostimulator with proximal facing primary fixating tines, and a set of proximally-mounted, proximally-biased secondary fixation tines 30. FIG. 13 shows a biostimulator with a primary fixation element in the form of distally-directed helix 20A, and generally proximally-directed convoluted tines serving as secondary fixating elements at the proximal end. Convoluted tines refer generally to a curved configuration with any level of complexity beyond that of a simple curve. FIGS. 12 and 13 also show the location of an electrode 68; as mentioned elsewhere, all embodiments include at least two electrodes, even though they are generally not shown in figures. FIG. 14 shows a biostimulator with proximally-directed primary fixating curved tines 20B at the distal portion of the device and two sets of proximally directed entangling tines 30 at two locations along the body of the biostimulator, at approximately the midsection and at the proximal end. FIG. 15 shows a biostimulator with a distally directed helix 20A and two sets of distally directed primary fixating straight tines 30 with end-knobs at two locations along the body of the biostimulator. FIG. 16 shows a biostimulator with a primary fixation element in the form of distally-directed helix 20A, a set of secondary fixating elements 30 in the form of a pair of distally directed clips mounted midway on the body of the biostimulator, and a set of straight tines with end-knobs at the distal portion, each set of secondary fixating elements mounted on a rotatable collar 65.

FIGS. 17A-17C show a series of embodiments of a leadless biostimulator 10, each with an active primary fixation element 20A at the distal end of the biostimulator, and each with a pair of passive secondary fixation elements 30 in the form of an entangling set of tines at the proximal portion and distal portion of the biostimulator. The entangling elements are biased and collapsible proximally, and may have varied proximal-facing angles when expanded as shown. The tines of FIG. 17A form an angle of about 90 degrees from the main axis of the biostimulator; the tines of FIG. 17B form an angle of about 45 degrees, and the tines of FIG. 17C form an angle of about 10 degrees. These embodiments reflect typical features of secondary fixation tines, as well as variations. What is typical is that secondary entangling elements 30 such as tines are generally biased proximally; this bias serves to have the orientation of the tines to generally conform-or be conformable to the surrounding ventricular walls, and it further precludes conflicting or interfering with interaction of a primary fixation element 20A, such as a screwable helix, with the primary attachment site 29. Angles at which the secondary fixating tines project from the main axis of a biostimulator may vary, as illustrated. The relative advantage of different project angles may be a function various factors, such as the linear location of the tines along the main axis, or the length of the tines, or the specifics of the shape and structure of the tines.

FIGS. 18A-18B show an embodiment of a leadless biostimulator 10 with an entangling set of tines 30 at the proximal portion of the biostimulator that are configured to serve as secondary fixation elements. FIG. 18A shows the tines collapsed proximally against the periphery of the biostimulator and secured in the collapsed position by a soluble biocompatible capsule 90. FIG. 18B shows the tines expanded into their deployed position, after the soluble capsule has dissolved. The use of a soluble biocompatible coating allows for sheathless deployment of a biostimulator, as has been described in US2007/0088418A1. The coating, previously described as a material to cover primary fixating elements, both active and passive, is also applicable to secondary fixating elements such as the proximally-situated and proximally-directed tines 30 of FIG. 18A. An exemplary material is mannitol, or other sugar derivatives, or polyvinylpyrrolidone, or a protective salt. Any biocompatible material that can be formed into a capsule as a dry form, and easily solubilized once exposed to an aqueous environment such as plasma, may be suitable. Upon dissolution of the capsule, typically after implantation of the biostimulator at its implant site, the capsule dissolves, and the tines expand to the deployed configuration, as seen in FIG. 18B.

FIGS. 19A-19B show an embodiment of a leadless biostimulator 10 with an entangling set of tines 30 at the proximal portion of the biostimulator that serve as secondary fixation elements and a primary fixation element in the form of a set of distally-mounted proximally angled tines. FIG. 19A shows both sets of tines collapsed distally against the periphery of the biostimulator and secured in the collapsed position by soluble capsules encasing both the proximal and distal ends of the biostimulator. FIG. 19B shows both sets of tines expanded into their deployed position, after the soluble capsule has dissolved.

FIG. 20 shows an embodiment of a leadless biostimulator 10 with a primary fixation element on the distal end, and secondary fixation elements in the form of proximally-facing entangling tines mounted on a rotatable collar encircling the biostimulator. The rotatability of the collar allows the body of the leadless biostimulator to rotate while a primary fixation element (such as a helix) engages the heart wall without interference from the secondary fixation element as it becomes entangled and its rotational movement stopped.

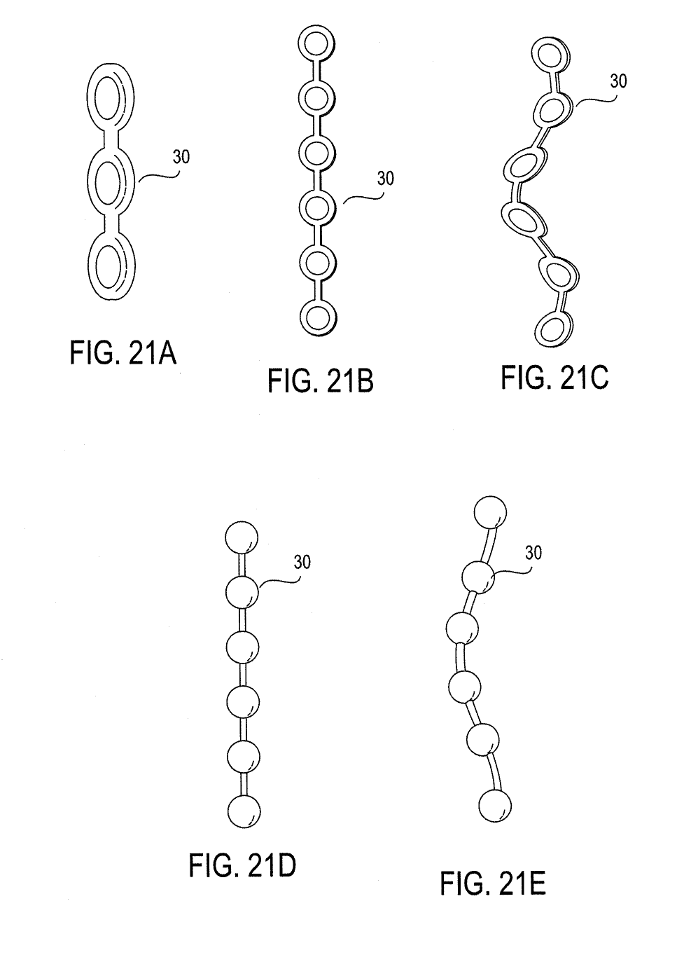

FIGS. 21A-21E shows several embodiments of entangling elements for secondary fixation of a leadless biostimulator 10, the entangling elements are variously knobbed, ringed, or beaded along a flexible spine, or linked together as in a chain. These embodiments may be considered variant embodiments of entangling tines. The flexibility of their spine or thread, or their flexibility as chain-like forms may advantageously enhance entangleability. These entangling embodiments may be attached to tines, directly on the body or housing of an LBS, or they may be mounted on a rotatable collar, as are typical entangling forms of secondary attachment elements.

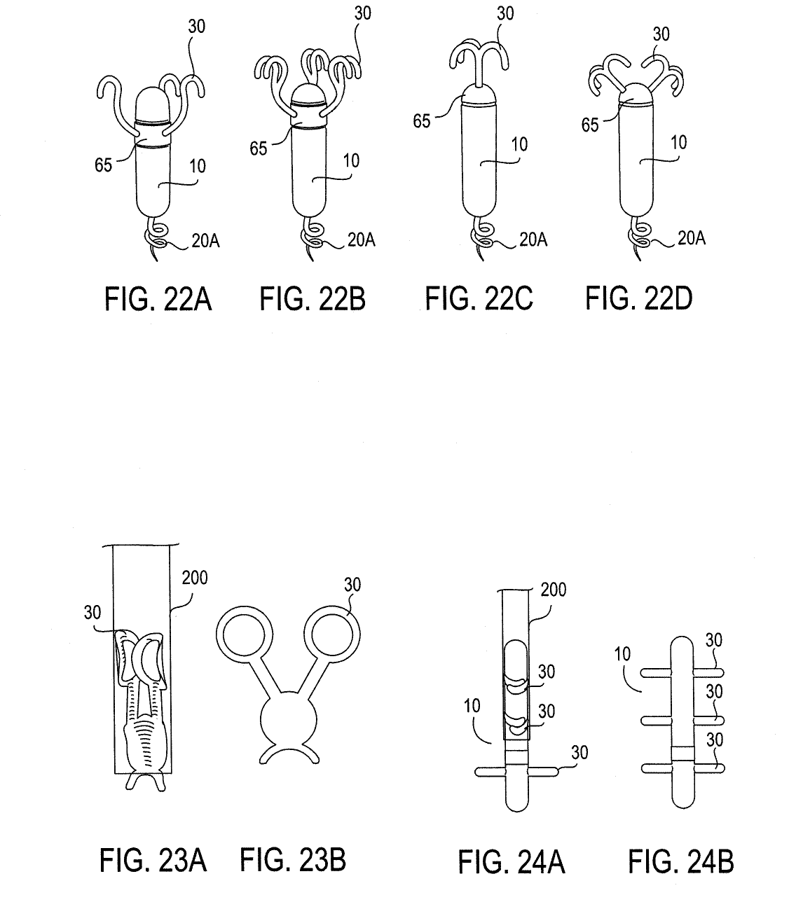

FIGS. 22A-22D show various fishhook-modified versions of secondary fixation tines. FIG. 22A shows a leadless biostimulator 10 with three fishhook-modified tines mounted on a rotatable collar at the distal portion of the device. FIG. 22B shows a similar leadless biostimulator embodiment, but with double fishhooks on each tine. FIG. 22C shows a leadless biostimulator with a single modified tine mounted on a rotating cap at the distal end of the device, the tine modified into a triple fishhook configuration. FIG. 22D shows a similar leadless biostimulator with multiple triple-hook modified tines. In various embodiments, these elements may be with tine structures, or attached to tines; attachments or junctions with tines may be variously fixed, bendable, or rotatable. Typically, the endpoints of the hook elements are atraumatic, their function is to snag, not necessarily to invade or embed. The tines, themselves, as in other embodiments of more simple tines, may be mounted on a rotatable collar that encircles the body or housing of a leadless biostimulator. The foregoing embodiments are provided as examples of a particular entangling element; other variations in terms of the number, precise configuration, and directionality of such elements are included as embodiments of the invention.

FIGS. 23A-23B show an example of a passive secondary fixation approach 20B in the form of ring-shaped entangling elements at the ends of tines with a distal-facing angle. Some examples of embodiments of this general form, when deployed, may form a lateral dimension sufficiently wide that movement through a ventricle exit such as the pulmonic valve is prevented in the event of detachment of the biostimulator from the primary fixation site. FIG. 23A depicts this embodiment compressed within a deployment tube, and FIG. 23B depicts the embodiment in a deployed state, the entangling or through-passage blocking elements in their expanded configuration.

FIGS. 24A-24B show an example of a secondary fixation approach which is similar to that represented by the embodiment shown in FIG. 23, in that entangling elements may occupy sufficient width that they preclude movement of a biostimulator 10 loosed from its primary attachment site through the pulmonic valve. FIG. 24A shows the biostimulator in a deployment tube; FIG. 24B shows the biostimulator in its post-deployment expanded configuration.

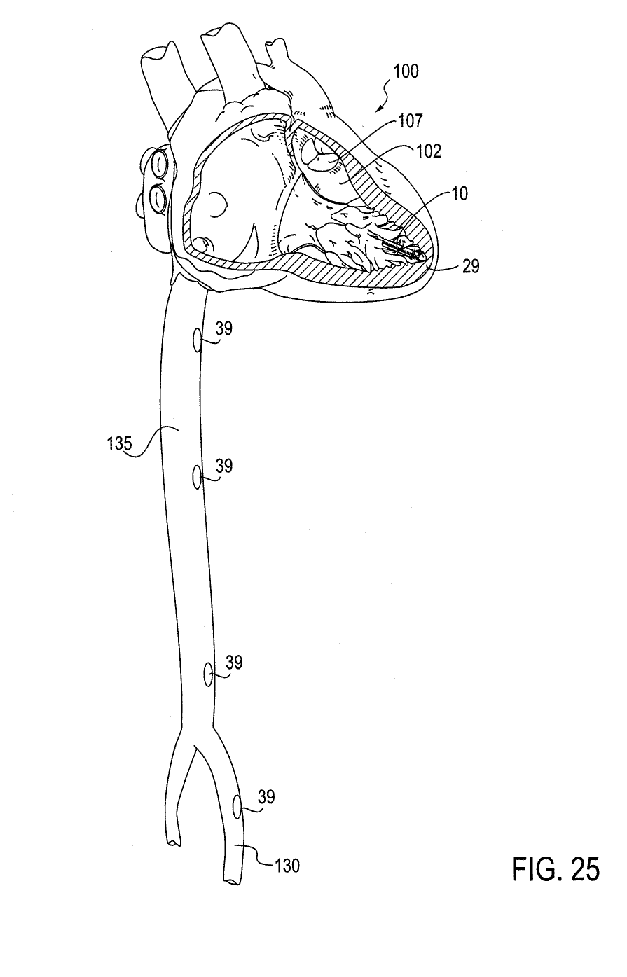

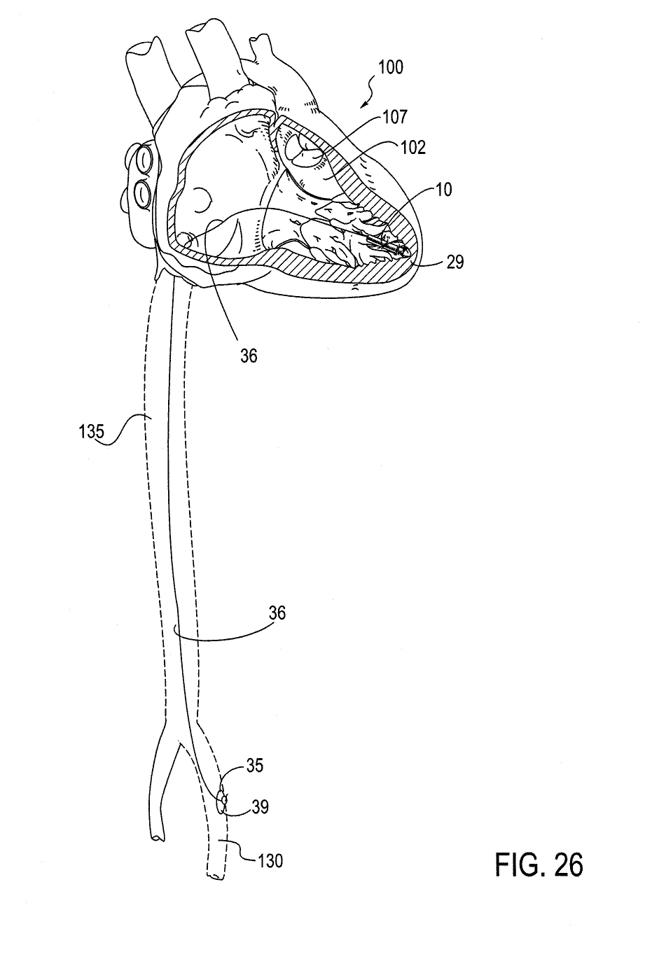

FIGS. 25-30 show biostimulators with embodiments of active secondary fixation systems that include an anchor 35 and a tether 36. FIG. 25 shows an embodiment of a leadless biostimulator 10 in situ at the apex of the right ventricle 102, further showing potential non-cardiac vascular sites 39 for anchoring a tether, these sites occur along the length of the inferior vena cava 135 and the femoral vein 130, which is a typical vascular path through which the biostimulator may be delivered to the implant site. FIG. 26 shows an embodiment of a leadless biostimulator 10 in situ at the apex of the right ventricle, and a tether 36 connecting the biostimulator 10 to an anchor 35 located at the left femoral vein 130.

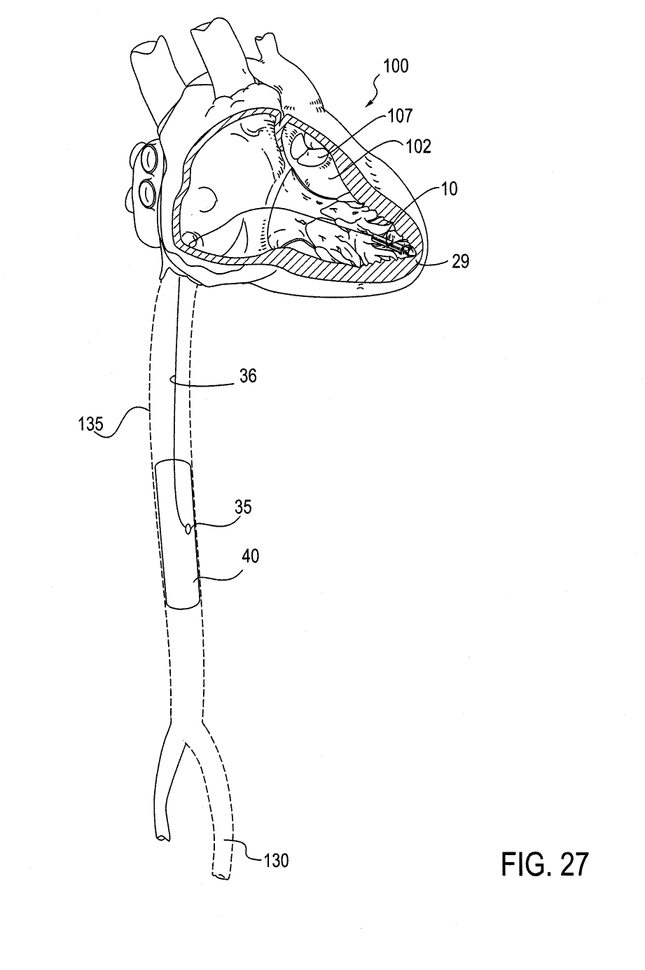

FIG. 27 shows an embodiment of a leadless biostimulator 10 in situ at the apex of the right ventricle, and a tether 36 connecting the biostimulator to an intraluminal stent 40 located within the inferior vena cava 135.

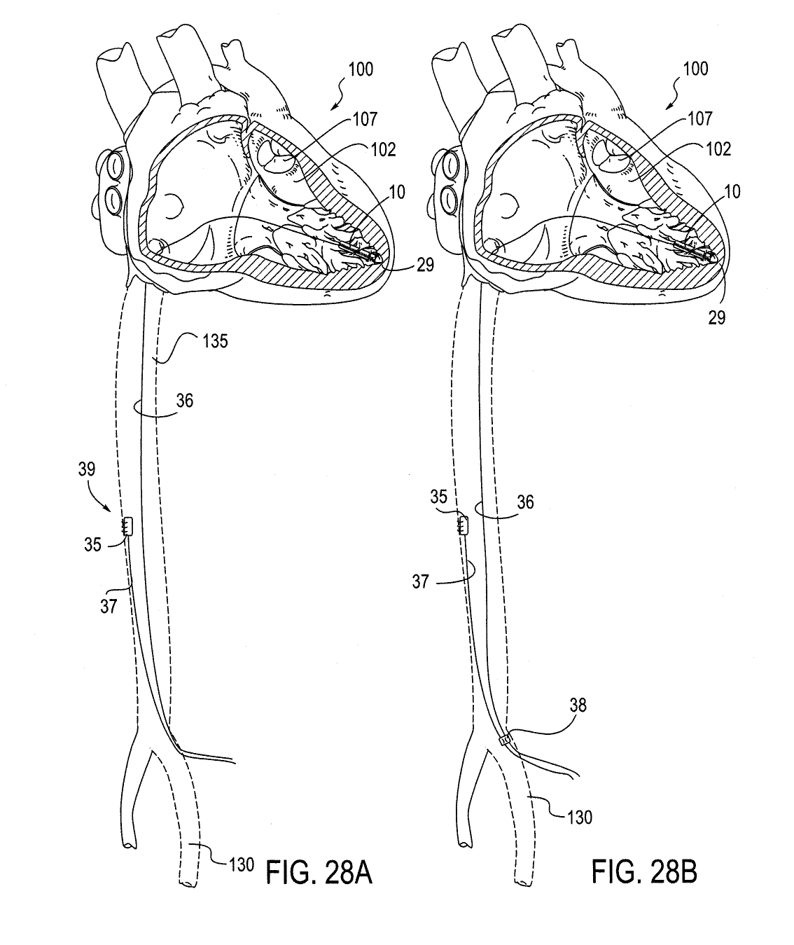

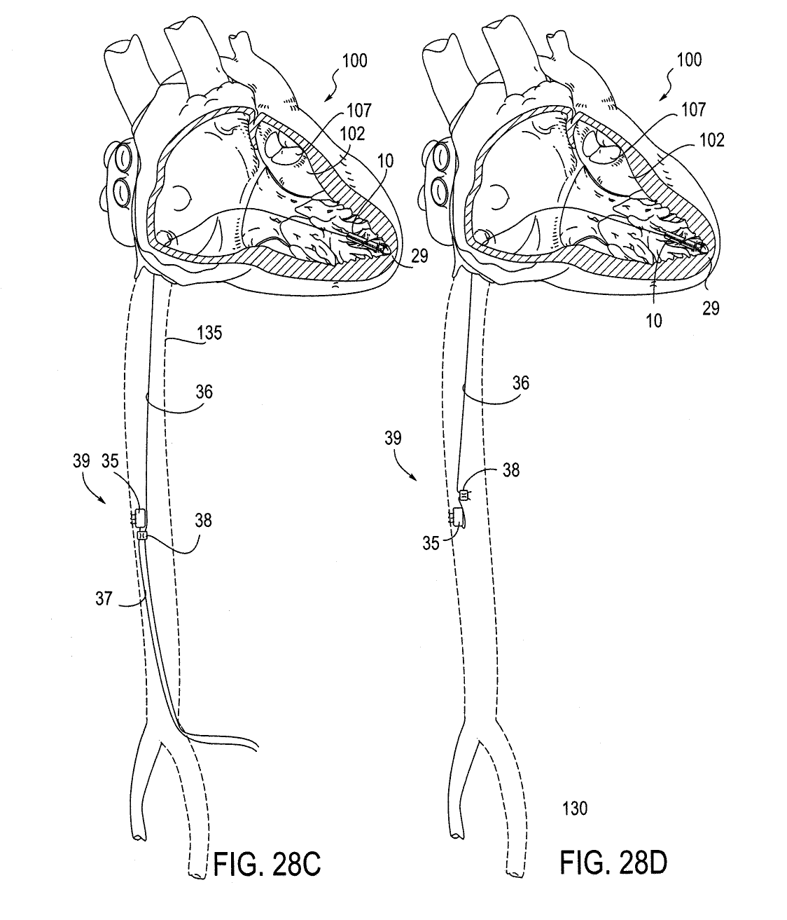

FIGS. 28A-28D show an embodiment of a leadless biostimulator 10 in situ at the apex of the right ventricle 102 with an alternatively-embodied actively fixating anchor-tether system, with the tether 36 connecting the biostimulator 10 to an anchoring site 39 located within the inferior vena cava 135. More particularly, FIGS. 28A-28D depict a method by which such a tether may be formed. FIG. 28A shows an early stage in the method, wherein a tether 36 proximally connected to the leadless biostimulator 10 emerges through a site in the femoral vein 130, and a second tether 37 proximally connected to an anchoring site along the length of the inferior vena cava 135 also emerges from the same site. In FIG. 28B, both tethers have been enclosed within a slidable clip 38, the clip is shown within the femoral vein 130 and is being advanced distally toward the anchoring site. In FIG. 28C, the clip has been distally advanced to the locale of the anchoring site, and the portions of each tether proximal to the clip are about to be cut off and removed, in order to form an integrated single tether. In FIG. 28D, the tether 36 formation is complete; it has become situated substantially proximal to the anchoring site and extends proximally toward the biostimulator 10 implanted and residing in the right ventricle 102, the clip 38 remaining at the junction of the formerly separate tethers.

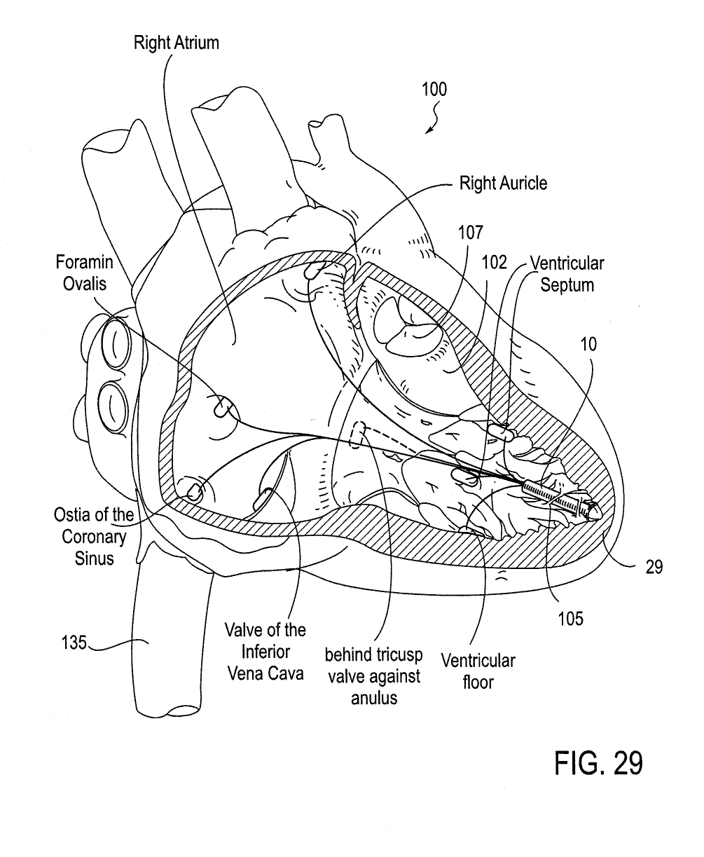

FIG. 29 shows an illustrative embodiment of a leadless biostimulator 10 with multiple active secondary fixation assemblies, each including an anchor 35 and a tether 36, the tether connecting the biostimulator 10 to various intracardial anchoring sites 39, the anchors located at various anchoring wall sites 39 within the right ventricle 102. The multiple sites are shown for purposes of illustration, any single embodiment might make use of any one or more of these anchoring sites.

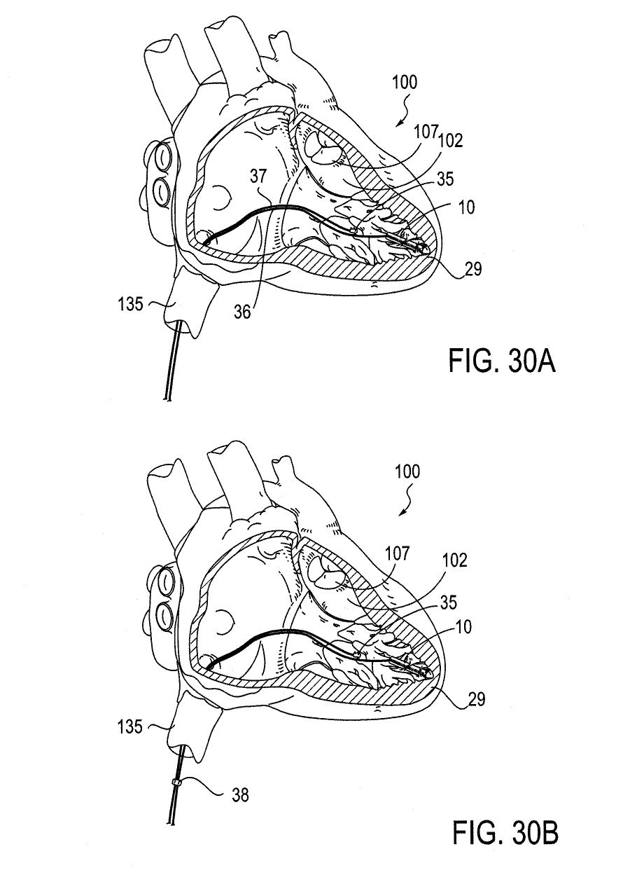

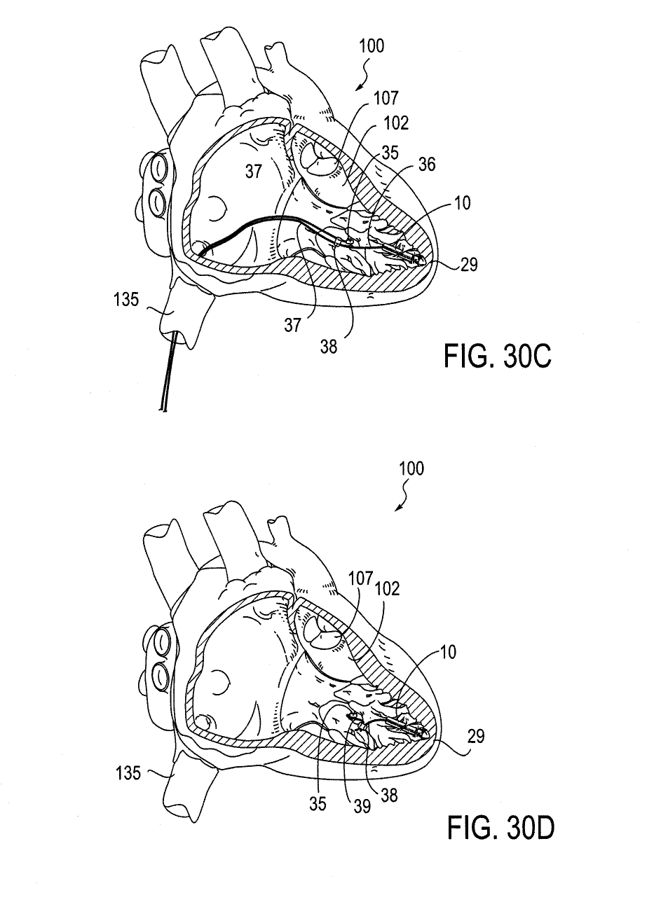

FIGS. 30A-30D show an embodiment of a leadless biostimulator 10 in situ at the apex of the right ventricle with an alternatively-embodied tether connecting the biostimulator to an anchoring site located within the right ventricle. This method is closely analogous to that described above and depicted in FIGS. 28A-28D, except that the secondary attachment site is different (intracardial vs. extracardial site), and except for the possible requirement for a differently configured tool for implanting the secondary anchor. FIG. 30A shows an early stage in the method, wherein a tethered biostimulator 10 with an attached tether 36 has been implanted in a ventricle 102, and a secondary anchor 35 with a secondary tether 37 has been implanted in the same ventricle. Both tethers exit the heart emerge from an entry/exit site in the femoral vein (not shown). In FIG. 30B, both tethers have been enclosed within a slidable clip 38, the clip is shown at a stage where it has been distally advanced from the entry site to a location in the inferior vena cava 135 and is about to enter the heart 100, more specifically the right ventricle 102. In FIG. 30C, the clip 38 has been distally advanced to the locale of the secondary fixation anchoring site 39, and the portions of each tether (36 and 37) distal to the clip are about to be cut off and removed, in order to form an integrated single tether. In FIG. 30D, the formation of the integrated tether 36 is complete; and it connects the biostimulator 10 directly to the anchoring site 39 on the ventricular wall.

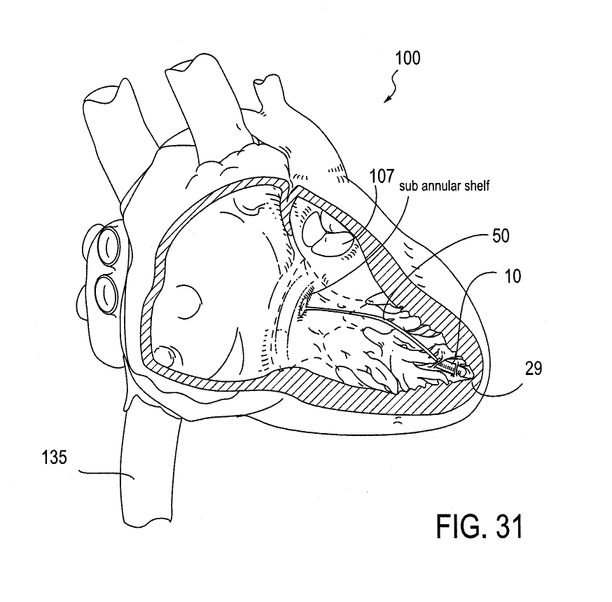

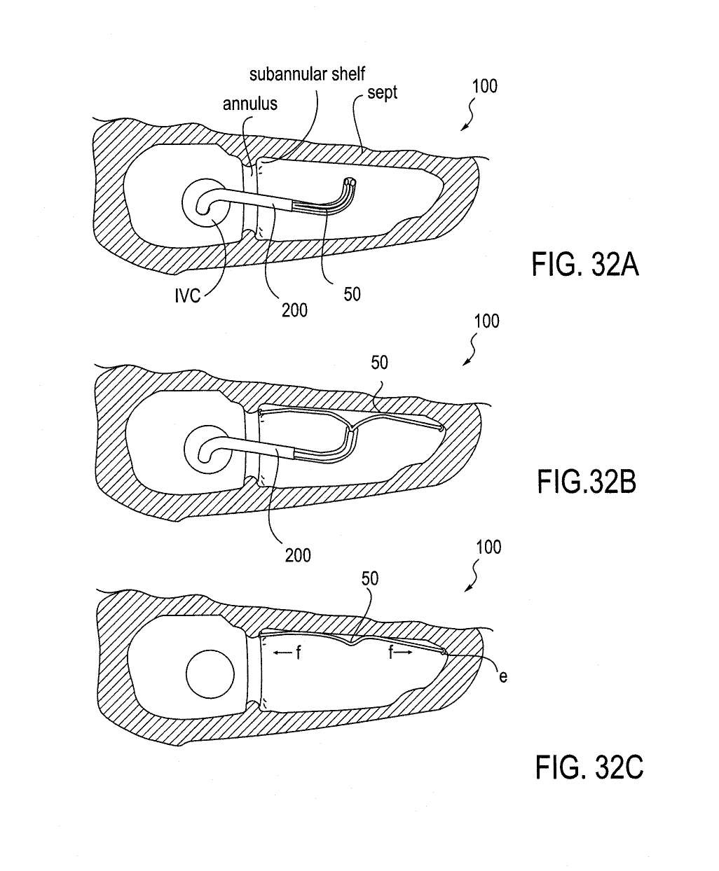

FIG. 31 shows an embodiment a leadless biostimulator with a flex member 50 that has expanded into a configuration as substantially rigid member that seats into the subannular shelf of the right ventricle. FIGS. 32A-32C show the deployment of the embodiment depicted in FIG. 31. FIG. 32A shows the flex member folded within a deployment tube about to emerge. FIG. 32B shows the flex member nearly completely emerged from the deployment tube 200, one of the ends seated against the subannular shelf, and the other seated against the proximal end of a leadless biostimulator at an implant site. FIG. 32C shows the expanded flex member in place. This embodiment of fixation may be described as a form of primary fixation that supports or enhances an already primarily fixated device, or it may also be understood as a redundant form of fixation, which supports maintaining the leadless biostimulator in a position such that intimate contact of at least one of the electrodes is maintained with the myocardium.

### Terms and Conventions

Unless defined otherwise, all technical terms used herein have the same meanings as commonly understood by one of ordinary skill in the art of cardiac technologies. Specific methods, devices, and materials may be described in this application, but any methods and materials similar or equivalent to those described herein can be used in the practice of the present invention. While embodiments of the invention have been described in some detail and by way of exemplary illustrations, such illustration is for purposes of clarity of understanding only, and understanding of the invention; it will be understood that the meaning of these various terms extends to common linguistic or grammatical variations or forms thereof. It will also be understood that when terminology referring to devices, equipment, or drugs that have been referred to by trade names, brand names, or common names, that these terms or names are provided as contemporary examples, and the invention is not limited by such literal scope. Terminology that is introduced at a later date that may be reasonably understood as a derivative of a contemporary term or designating of a hierarchal subset embraced by a contemporary term will be understood as having been described by the now contemporary terminology. Further, while some theoretical considerations have been advanced in furtherance of providing an understanding of the invention, the claims to the invention are not bound by such theory. Moreover, any one or more features of any embodiment of the invention can be combined with any one or more other features of any other embodiment of the invention, without departing from the scope of the invention. Still further, it should be understood that the invention is not limited to the embodiments that have been set forth for purposes of exemplification, but is to be defined only by a fair reading of claims that are appended to the patent application, including the full range of equivalency to which each element thereof is entitled.

What is claimed is:

1. A leadless biostimulator comprising:
a power source adapted to be disposed within a human heart chamber;
an electrode in electrical communication with the power source and adapted to be placed in contact with tissue within the heart chamber;
a controller adapted to be disposed within the heart chamber and to control delivery of electrical energy from the power source to the electrode;
a primary fixation element adapted to affix the biostimulator to a primary fixation site on a heart wall within the heart chamber; and
a downstream vascular escape prevention assembly adapted to prevent an escape of the biostimulator in the event of it being dislodged from the primary fixation site.

2. The leadless biostimulator of claim 1 further comprising a housing in which the power source, the electrode, and the controller are disposed.

3. The leadless biostimulator of claim 1 wherein the heart chamber into which the biostimulator is adapted to be implanted is any of the right ventricle, left ventricle, right atrium, or left atrium.

4. The leadless biostimulator of claim 1 wherein the downstream vascular escape prevention assembly comprises one or more entangling elements adapted to entangle within heart structure at one or more secondary fixation sites within the chamber of the heart.

5. The leadless biostimulator of claim 4 wherein the one or more entangling elements comprise any of tines, hooks, or chains.

6. The leadless biostimulator of claim 4 wherein the entangling elements are adapted to extend radially outward beyond the diameter of the biostimulator when implanted within the heart chamber.

7. The leadless biostimulator of claim 4 wherein the entangling elements are at least 5 mm in length.

8. The leadless biostimulator of claim 4 wherein the entangling elements extend outward from the biostimulator at a proximal-facing angle that ranges from about 10 degrees to about 90 degrees from the axis of the biostimulator.

9. The leadless biostimulator of claim 4 wherein the tines are configured as any of straight tines, curvilinear tines, or convoluted tines.

10. The leadless biostimulator of claim 4 wherein the entangling elements are adapted to be rotatable with respect to the biostimulator.

11. The leadless biostimulator of claim 10 wherein the entangling elements are mounted on a rotatable collar encircling the main axis of the biostimulator.

12. The leadless biostimulator of claim 4 wherein the entangling elements are configured such that they are distally collapsible around the periphery of the biostimulator.

13. The leadless biostimulator of claim 12 wherein the collapsible entangling elements, when collapsed, are configured to be substantially contained within a maximal diameter of the biostimulator.

14. The leadless biostimulator of claim 1 wherein the downstream vascular escape prevention assembly comprises a tether and an anchor adapted to anchor at a secondary attachment site, the tether connecting the assembly and the anchor to each other.

15. The leadless biostimulator of claim 14 wherein the anchor includes comprises a screw, a hook, a clip, a stent, a cage, and/or a barb adapted to attach the biostimulator to the secondary attachment site.

16. The leadless biostimulator of claim 14 wherein the secondary attachment site may be any of an intracardiac site, an intravascular site, or an extravascular site.

17. The leadless biostimulator of claim 16 wherein the intracardiac site is a septal wall of the heart.

18. The leadless biostimulator of claim 16 wherein the intravascular site is located within a vessel through which the biostimulator is adapted to be delivered to the heart.

19. The leadless biostimulator of claim 18 wherein the vessel includes any of the femoral vein or the inferior vena cava.

20. The leadless biostimulator of claim 18 wherein the tether is formed from two segments secured together with a clip.

21. The leadless biostimulator of claim 16 wherein the extravascular site includes the external periphery of a vessel through which the biostimulator was delivered to the heart.

22. The leadless biostimulator of claim 21 wherein the tether is adapted to be threaded through the vessel wall and is attached to the anchor, the anchor comprising any of a partial cylinder, a plate, and/or a ball.

23. The leadless biostimulator of claim 14 wherein the anchor comprises one or more electrodes for biostimulation, and wherein the tether is electrically conductive.

24. The leadless biostimulator of claim 14 wherein the tether comprises any of single strand wire, multistranded wire, monofilament suture thread, or multistrand suture thread.

25. The leadless biostimulator of claim 14 wherein the tether comprises a biodegradable material.

26. The leadless biostimulator of claim 14 wherein the tether comprises an antithrombogenic agent.

27. The leadless biostimulator of claim 1 further comprising one or more soluble coverings configured to encapsulate any of the primary fixation element or the secondary fixation element.

28. The leadless biostimulator of claim 27 wherein the soluble covering is biocompatible.

29. The leadless biostimulator of claim 27 wherein the soluble covering comprises any of a polymer, a protective sugar, or a protective salt.

30. The leadless biostimulator of claim 29 wherein the protective sugar is mannitol.

31. The leadless biostimulator of claim 29 wherein the polymer is polyvinylpyrrolidone.

32. The leadless biostimulator of claim 27 wherein the secondary element is collapsible around the periphery of the biostimulator, and wherein the soluble covering secures the secondary element in a collapsed configuration.

33. A method for retaining a leadless intracardiac biostimulator in a heart in the event of dislodgement from a primary fixation site comprising:
entangling an element of the biostimulator within the heart structure at a secondary fixation site within a heart chamber, such entanglement being sufficient to retain the biostimulator within the cardiac chamber.

34. The method of claim 33 wherein entangling an element of the biostimulator within a heart structure comprises entangling the element within a structure in the left ventricle.

35. The method of claim 33 wherein entangling an element of the biostimulator within a heart structure comprises entangling the element within a structure in the right ventricle.

36. The method of claim 33 further including preventing escape of the biostimulator into a downstream vascular site.

37. The method of claim 36 wherein preventing escape of the biostimulator into a downstream vascular site comprises preventing escape into the pulmonary artery.

38. The method of claim 36 wherein preventing escape of the biostimulator into a downstream vascular site comprises preventing escape into the aorta.

39. A method for retaining a leadless intracardiac biostimulator in a heart in the event of dislodgement from a primary fixation site comprising:
anchoring the biostimulator with a tether to a secondary fixation site, the tether being of appropriate length to prevent substantial movement of the biostimulator into a downstream vascular from the primary fixation site of the biostimulator in a heart chamber.

40. The method of claim 39 wherein anchoring the biostimulator with a tether comprises anchoring the biostimulator with a tether of appropriate length to retain the biostimulator within the heart chamber.

41. The method of claim 39 wherein anchoring the biostimulator with a tether comprises attaching the tether to an anchor at the secondary fixation site.

42. The method of claim 41 wherein anchoring comprises attaching the tether to the secondary fixation site with any of a screw, a hook, a clip, a stent, a cage, or a barb.

43. The method of claim 39 wherein anchoring the biostimulator to a secondary fixation site comprises anchoring to any of an intracardiac site or an extracardial site.

44. The method of claim 43 wherein anchoring to an extracardial site comprises anchoring to a site on a vessel through which the biostimulator was delivered to the heart.

45. The method of claim 44 wherein the anchoring to a site on a vessel through which the biostimulator was delivered to the heart comprises anchoring to a site on any of the internal or exterior surface of the vessel.

46. The method of claim 39 wherein anchoring with a tether comprises combining two tethers to form a single tether, the method comprising:
inserting the biostimulator attached to a first tether into an entry site in the vasculature, advancing the biostimulator to an intracardial implant site, and implanting the biostimulator at that site;
inserting a secondary anchor attached to a second tether into the entry site in the vasculature, advancing the anchor to the secondary fixation site, and implanting the anchor at that site; and
engaging the tether of the biostimulator and the tether of the anchor within a slidable clip at the vascular entry site to form a combined tether.

47. The method of claim 46 further comprising:
adjusting the length of the combined tether by slidably advancing the clip within the vasculature toward the secondary fixation site; and
securing the first tether and the second tether at the clip so that no further sliding can occur.

48. The method of claim 47further comprising removing remnant lengths of the first tether and second tether that extend from the clip through the vasculature entry site.

49. The method of claim 47 wherein adjusting the length of the tether includes removing slack in the tether.
